(19) United States
(12) Patent Application Publication
(10) Pub. No.: US 2023/0240922 A1
(43) Pub. Date: Aug. 3, 2023

(54) BED WITH FEATURES FOR DETERMINING RISK OF CONGESTIVE HEART FAILURE

(71) Applicant: Sleep Number Corporation, Minneapolis, MN (US)

(72) Inventors: Megha Rajam Rao, San Jose, CA (US); Gary N. Garcia Molina, Madison, WI (US)

(21) Appl. No.: 18/104,634

(22) Filed: Feb. 1, 2023

Related U.S. Application Data
(60) Provisional application No. 63/305,713, filed on Feb. 2, 2022.

(51) Int. Cl. A61G 7/05; A61B 5/11; A61B 5/00; G16H 40/67; G16H 50/30; G16H 50/70

(52) U.S. Cl. CPC A61G 7/05; A61B 5/1102; A61B 5/746; A61B 5/4809; G16H 40/67; G16H 50/30; G16H 50/70

(57) ABSTRACT

Disclosed are techniques for determining a probability measurement of cardiac failure risk (e.g., congestive heart failure or CHF) for a user of a bed system. A method can include receiving, by a computer system, user data collected by sensors of a bed system when a user rests on the bed system, determining sleep fragmentation information for a sleep session of the user based on analyzing the user data, providing the sleep fragmentation information as input to a model that was trained to determine a probability measurement of a risk of cardiac failure for users of bed systems based at least in part on user data associated with the users, receiving the probability measurement of the risk of cardiac failure for the user of the bed system as output from the model, and performing an action based on the probability measurement.

### INCORPORATION BY REFERENCE

This application claims priority to U.S. Provisional Application Ser. No. 63/305,713, filed on Feb. 2, 2022, the disclosure of which is incorporated by reference in its entirety.

### TECHNICAL FIELD

This document describes devices, systems, and methods related to a bed capable of predicting congestive heart failure and cardiac failure in users on the bed.

### BACKGROUND

In general, a bed is a piece of furniture used as a location to sleep or relax. Many modern beds include a soft mattress on a bed frame. The mattress may include springs, foam material, and/or an air chamber to support the weight of one or more occupants.

### SUMMARY

The document generally relates to systems, methods, and techniques for predicting risk of congestive heart failure (CHF) and/or other cardiac failure in users of bed systems, such as smart beds. More particularly, the disclosed technology provides for unobtrusively monitoring cardiovascular activity of users of bed systems to determine indices of their sleep fragmentation and abnormal patterns in cardiovascular activity to generate an index (e.g., a probability, a likelihood, and/or a measurement) of cardiac failure risk. The index of cardiac failure risk can be generated over time (e.g., over multiple sleep sessions) and/or per any given night. Data collected about a user by a bed system can be provided as input to a model that was trained using machine-learning techniques to determine probability measurements of CHF risk or other cardiac failure for the user. A variety of data can be collected and used to accurately assess risk of developing CHF in different users of bed systems. For example, the probability measurement of risk of CHF can be based on sleep fragmentation information of a user. The probability measurement can also be based on other data, including but not limited to sleep and/or wake detection, wake after sleep onset, wake before sleep onset, sleep efficiency, sleep duration, sleep-bout duration, time to fall asleep, arousal index, heartrate (HR), respiratory rate (RR), daytime alertness, demographic factors, blood pressure, and/or inter-beat intervals (IBI). The disclosed technology can therefore be used to quantify risk of CHF as a probability measurement, not merely identification of a risk of CHF. A logistic regression model can be used to determine the probability measurement.

CHF can describe situations in which a user's heart fails when it does not pump blood normally. CHF can be categorized as acute or chronic. Acute CHF may occur suddenly and can lead to a medical emergency while chronic CHF can develop gradually over time. CHF can be a marker for deterioration of cardiac health. For instance, it can cause the heart to enlarge or beat faster, which can cause the heart to weaken and lower its pump efficiency over time. Moreover, due to heart muscle inability, there can be a backlog of blood returned to the heart, thereby causing congestion. Oxygen levels may drop in cells of the user's body, fluid can build up in the lungs, and the user can experience heart palpitations and/or shortness of breath. CHF can have grave impacts on the user's overall health, wellbeing, and organs.

Sleep fragmentation information can be used as input to the models described herein to generate risk probability measurements of CHF in users of bed systems. Sleep fragmentation can be defined as repeated, short sleep interruptions during a sleep session (and/or sleep cycle), which can cause excessive tiredness during following days. Sleep fragmentation can affect a user's natural sleep session and/or sleep stages and may be a symptom of a sleep disorder. Moreover, sleep fragmentation can be quantified based on data that can be sensed by a bed system, such as wake after sleep onset, wake before sleep onset, arousal index, and alertness levels.

One or more embodiments described herein can include a method for determining a probability measurement of a risk of cardiac failure for a user of a bed system, the method including: receiving, by a computer system, user data collected by sensors of a bed system when a user rests on the bed system, determining, by the computer system, sleep fragmentation information for a sleep session of the user based on analyzing the user data, providing, by the computer system, the sleep fragmentation information as input to a model that was trained to determine a probability measurement of a risk of cardiac failure for users of bed systems based at least in part on user data associated with the users, receiving, by the computer system, the probability measurement of the risk of cardiac failure for the user of the bed system as output from the model, and performing, by the computer system, an action based on the probability measurement.

In some implementations, the embodiments described herein can optionally include one or more of the following features. For example, the user data can include ballistocardiogram (BCG) signals. The model could have been trained with a training dataset of historic user data associated with the user. The model could have been trained with a training dataset of historic user data associated with different users of the bed systems. The model can be a logistic regression model. The model could have been trained, by the computer system, using feature vectors that were assigned numeric values corresponding to training user data. The training user data can include at least one of wake time after sleep onset, sleep efficiency, sleep duration, and sleep-bout duration. The training user data can also include at least one of wake time before sleep onset, quantity of sleep disruptions, heartrate, heartrate variability, respiratory rate, and daytime alertness levels. The training user data may also include demographics information, the demographics information including at least one of weight, age, gender, and body mass index (BMI).

In some implementations, determining, by the computer system, sleep fragmentation information for the user can include identifying periods of time during the sleep session when the user is sleeping and periods of time during the sleep session when the user is awake. In some implementations, the method can also include providing, by the computer system, at least one of (i) wake after sleep onset data, (ii) sleep efficiency data, (iii) sleep duration data, and (iv) sleep-bout duration data as input to the model. The model could have been trained, by the computer system, to correlate at least one of (i)-(iv) with stages of sleep fragmentation for the users of the bed systems. Moreover, the probability measurement of the risk of cardiac failure can be a numeric value.

In some implementations, the output from the model can be a value between 0 and 1, and the method can also include: multiplying, by the computer system, the value with a numeric factor to generate a score of the risk of cardiac failure. Sometimes, the probability measurement of the risk of cardiac failure can be a categorical value, the categorical value being at least one of low risk, medium risk, and high risk.

In some implementations, performing, by the computer system, an action based on the probability measurement can include outputting an alert at a user device of the user indicating that the user is at risk of cardiac failure based on a determination that the probability measurement exceeds a threshold range. Moreover, performing, by the computer system, an action based on the probability measurement can include transmitting a notification to a user device of a healthcare provider that the user is at risk of cardiac failure based on a determination that the probability measurement exceeds a threshold range. Additionally, the cardiac failure can be congestive heart failure.

One or more embodiments described herein can include a method for determining a probability measurement of a risk of cardiac failure for a user of a bed system, the method including: receiving, by a computer system, user data collected by sensors of a bed system when a user rests on the bed system, determining, by the computer system, sleep fragmentation information for a sleep session of the user based on analyzing the user data, determining, by the computer system and based on analyzing the user data, at least one of (i) wake after sleep onset data, (ii) sleep efficiency data, (iii) sleep duration data, and (iv) sleep-bout duration data, providing, by the computer system, the sleep fragmentation information and at least one of (i)-(iv) as input to a model that was trained to determine a probability measurement of a risk of cardiac failure for users of bed systems based at least in part on user data associated with the users, receiving, by the computer system, the probability measurement of the risk of cardiac failure for the user of the bed system as output from the model, and performing, by the computer system, an action based on the probability measurement.

One or more embodiments described herein can also include a system for determining a probability measurement of a risk of cardiac failure for a user of a bed system, the system having: a bed having at least one sensor and a computer system in communication with the at least one sensor of the bed. The computer can be configured to: receive, from the at least one sensor, user data collected by the at least one sensor when a user rests on the bed, determine sleep fragmentation information for a sleep session of the user based on analyzing the user data, provide the sleep fragmentation information as input to a model that was trained to determine a probability measurement of a risk of cardiac failure for users of beds based at least in part on user data associated with the users, receive the probability measurement of the risk of cardiac failure for the user of the bed as output from the model, and perform an action based on the probability measurement.

The system can optionally include one or more of the following features. For example, the bed further can include a controller, and the computer system can be the controller. The computer system can also be remote from the bed.

One or more embodiments described herein can include a system for determining a probability measurement of a risk of congestive heart failure for a user of a bed system, the system including a bed having at least one sensor and a computer system in communication with the at least one sensor of the bed. The computer system can: determine sleep fragmentation as a function of sensed data from the at least one sensor of a sleeper of the bed, calculate a probability measurement of congestive heart failure as a function of the determined sleep fragmentation, and output a signal to perform an action based on the calculated probability measurement of congestive heart failure.

One or more embodiments described herein can also include a system for determining a probability measurement of a risk of cardiac failure for a user of a bed system, the system including: a bed having at least one sensor and a computer system in communication with the at least one sensor of the bed, the computer system being configured to: receive, from the at least one sensor, user data collected by the at least one sensor when a user rests on the bed, determine sleep fragmentation information for a sleep session of the user based on analyzing the user data, determine at least one of (i) wake after sleep onset data, (ii) sleep efficiency data, (iii) sleep duration data, and (iv) sleep-bout duration data based on analyzing the user data, provide the sleep fragmentation information and at least one of (i)-(iv) as input to a model that was trained to determine a probability measurement of a risk of cardiac failure for users of beds based at least in part on user data associated with the users, receive the probability measurement of the risk of cardiac failure for the user of the bed as output from the model, and perform an action based on the probability measurement.

One or more embodiments described herein can include a system for determining a probability measurement of a risk of congestive heart failure for a user of a bed system, the system having: a bed having at least one sensor and a computer system in communication with the at least one sensor of the bed, the computer system being configured to: determine wake after sleep onset data as a function of sensed data of a sleeper from the at least one sensor, determine sleep efficiency data as a function of the sensed data from the sleeper, determine sleep-bout duration data as a function of the sensed data from the sleeper, calculate a probability measurement of congestive heart failure as a function the determined wake after sleep onset data, sleep efficiency data, and sleep-bout duration data, and output a signal to perform an action based on the calculated probability measurement of congestive heart failure.

The devices, system, and techniques described herein may provide one or more of the following advantages. For example, the disclosed technology leverages sleep and detection capabilities of bed systems (e.g., smart beds with sensors) to quantify sleep fragmentation and determine probability measurements of CHF risk in users of the bed systems. Using existing technology, users of bed systems can gain greater insight into their health, and more specifically their heart health, without having to invest in additional technology. Sensors, for example, that may be used to measure pressure changes in air chambers of the bed system can also be purposed for measuring pressure changes useful for determining sleep fragmentation information about the user.

Moreover, the disclosed technology leverages machine-learning techniques to provide for accurate assessment of a variety of data to determine the probability measurements of CHF risk in various users. The disclosed technology can leverage a variety and abundance of data (both historic and real time data about a particular user and/or groups of users), including various different data types and data from a variety of sources, to more accurately determine the probability measurements. For example, system-based determinations and predictions (e.g., determined alertness levels) can be combined with raw sensor data (e.g., BCG signals) to determine the probability measurements both accurately and quickly for many users having different health conditions, demographic factors, and other unique attributes. The machine-learning models can also be continuously trained and improved as more data is collected by the bed systems and more probability measurement determinations are made for different users. As a result, CHF risk in different users can be accurately predicted and assessed over time as well as in real time.

As another example, the disclosed technology provides for unobtrusively and automatically monitoring the user's health while they are sleeping, and also awake, to glean additional insights about the user's health over time and/or during a sleep session. As a result, the user may not have to remember or keep track of changes in their health or needing to schedule visits with physicians to do routine checkups or random checkups if the user is feeling uncertain about their cardiac health. Furthermore, if a medical concern is discerned using the disclosed technology, the user, as well as relevant healthcare providers, can have access to months worth of user data, even before the medical concern appeared, was detected, or otherwise became known. This abundance of user data can help the user and/or the relevant healthcare providers understand how the user's medical concern developed and/or how to treat or otherwise prevent the medical concern from greatly impacting the user's life.

Moreover, a user may go to a doctor's office to test the user's cardiac health at various times throughout their life (e.g., monthly, yearly, every couple of months, every couple of years, etc.). Such short visits may not accurately capture cardiovascular activity of the user, especially since the user is typically awake and their body is focused on many tasks rather than being relaxed and at rest. The disclosed technology, therefore, can be used to measure and quantify cardiovascular activity over many sleep sessions when the user's body is relaxed and at rest to accurately determine cardiac health of the user. Data can be collected about the user while their body is at rest and over extended periods of time. The ability to monitor the user's health during sleep can provide more accurate health detections and lower a need for the user to see a physician to check their health and cardiac metrics. This can also be beneficial to lower healthcare costs for the user, especially if the user does not have insurance coverage for cardiac health testing and care. Anomalies in cardiovascular activity can be more accurately and acutely identified and assessed when multisensory data is dynamically being collected about the user over extended periods of time and fed into a model trained to measure cardiac health.

Similarly, the disclosed technology provides for determining probability measurements of CHF risk in users on a nightly basis for prolonged periods of time. This can provide for accurate and frequent quantification of heart health in comparison to sleep clinic polysomnography (PSG) recordings, which, due to costs and patient burden, may only happen once in a few months or even years.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram for determining a probability measurement of congestive heart failure in a user of a bed system.

FIG. 2 is a conceptual diagram for training a model to determine a probability measurement of congestive heart failure in a user of a bed system.

FIG. 3 is a flowchart of a process for determining a probability measurement of congestive heart failure in a user of a bed system.

FIG. 4 is a system diagram of one or more components that can be used to perform the disclosed techniques.

FIG. 5 is a schematic diagram that shows an example of a computing device and a mobile computing device.

Like reference symbols in the various drawings indicate like elements.

### DETAILED DESCRIPTION OF ILLUSTRATIVE EMBODIMENTS

This document generally relates to techniques for estimating risk of cardiac health deterioration, such as CHF, in users of bed systems. More particularly, multisensory data collected by sensors (e.g., pressure sensors, temperature sensors, motion sensors, audio sensors, etc.) of a bed system (e.g., smart bed) can be combined with data insights determined by a computing system about a user of the bed system to determine a probability measurement of risk of CHF in the user. The computing system can use machine-learning trained models to determine the probability measurement based on a variety of data points. For example, the computing system can collect data measured by a bed system throughout a user's sleep session. Using the collected data, the computing system can estimate a coefficient that may quantify CHF risk for the particular user of the bed system. The coefficient can, for example, be based on sleep fragmentation information determined by the computing system and based on the collected data during a sleep session or over time (e.g., over several sleep sessions). The coefficient can then be provided, by the computing system, as input to a machine-learning and/or AI-based model to quantify CHF risk. As described herein, the model can determine a probability measurement of CHF risk in the particular user. Based on the probability measurement determined using the model, the computing system may generate output, alerts, and/or notifications to be presented at user devices of the user of the bed system, healthcare providers, or other relevant users.

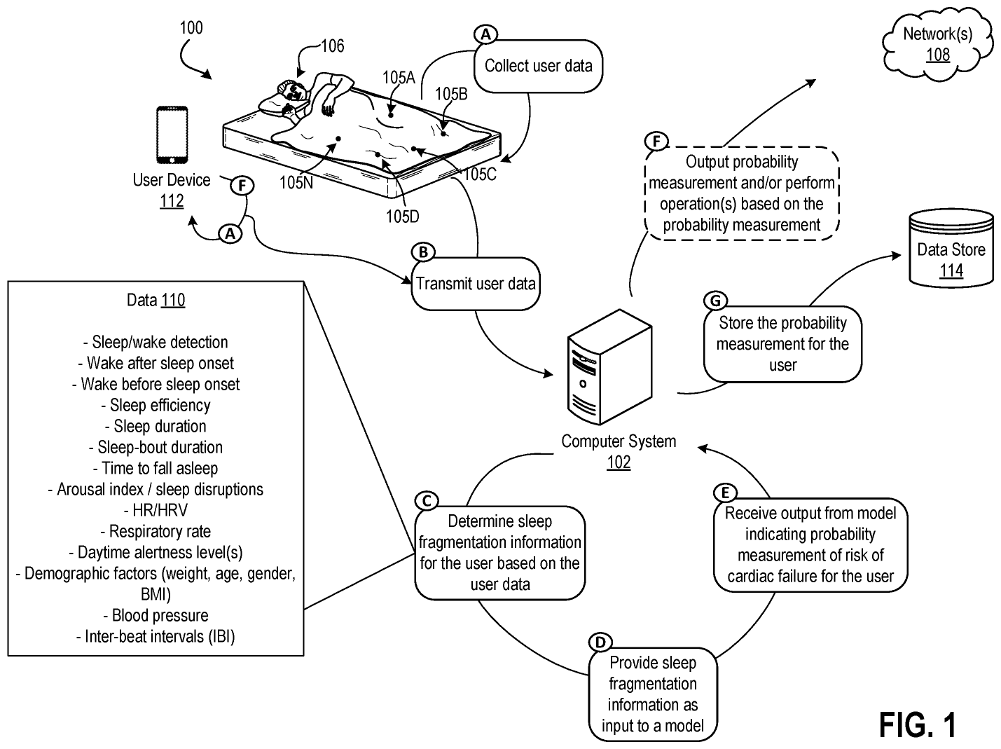

Referring to the figures, FIG. 1 is a conceptual diagram for determining a probability measurement of congestive heart failure in a user 106 of a bed system 100. The bed system 100, computer system 102, user device 112, and data store 114 can communicate via network(s) 108. The network(s) 108 can provide wired and/or wireless communication between components described herein (e.g., BLUETOOTH, Internet, WIFI, Local Area Network, etc.).

In brief, the bed system 100 can be a smart bed. The bed system 100 can include one or more sensors 105A-N. The sensors 105A-N can be arranged in various layouts and/or arrays throughout the bed system 100. For example, one or more of the sensors 105A-N can be configured to or otherwise integrated into a foundation, side rails, headboard, and/or legs of the bed system 100. One or more sensors 105A-N can, for example, be load cells positioned in each of the legs of the bed system 100 and configured to measure weight and/or pressure changes (e.g., fine-grained weight changes) in the bed system 100. One or more of the sensors 105A-N can also include pressure sensors integrated into the bed system 100, such as in a mattress and/or pump system that is fluidically connected to air chambers of the mattress (where the mattress is an air mattress or other inflatable mattress). One or more of the sensors 105A-N can also include temperature sensors. In some implementations, one or more of the sensors 105A-N can include audio sensors configured to detect sound within a predetermined decibel range for identification of snore or other sleep-related activities. One or more other types of sensors can be used in the bed system 100. Moreover, one or more of the sensors 105A-N can be part of a pad, mat, or other material that overlays a portion of the bed system 100, such as a pad that covers a portion of the mattress.

The bed system 100 can include one or more air chambers that can receive fluids such as air or liquid for inflation and/or deflation of such air chambers. Pressure sensors can be positioned within the air chambers. The pressure sensors can also be fluidly connected to the air chambers, such as along air tubes. The air chambers can be in fluid communication with a pump of the bed system 100, which can be in electrical communication with a remote control and/or the user device 112 via a control box. The control box can, for example, operate the pump to cause increases and decreases in the fluid pressure of the air chambers based upon commands input by the user 106 using the remote control or their user device 112. Pressure changes and other data can be collected from sensors 105A-N that may be placed, positioned, or otherwise integrated into the air chambers, pump, and/or other portions of the bed system 100.

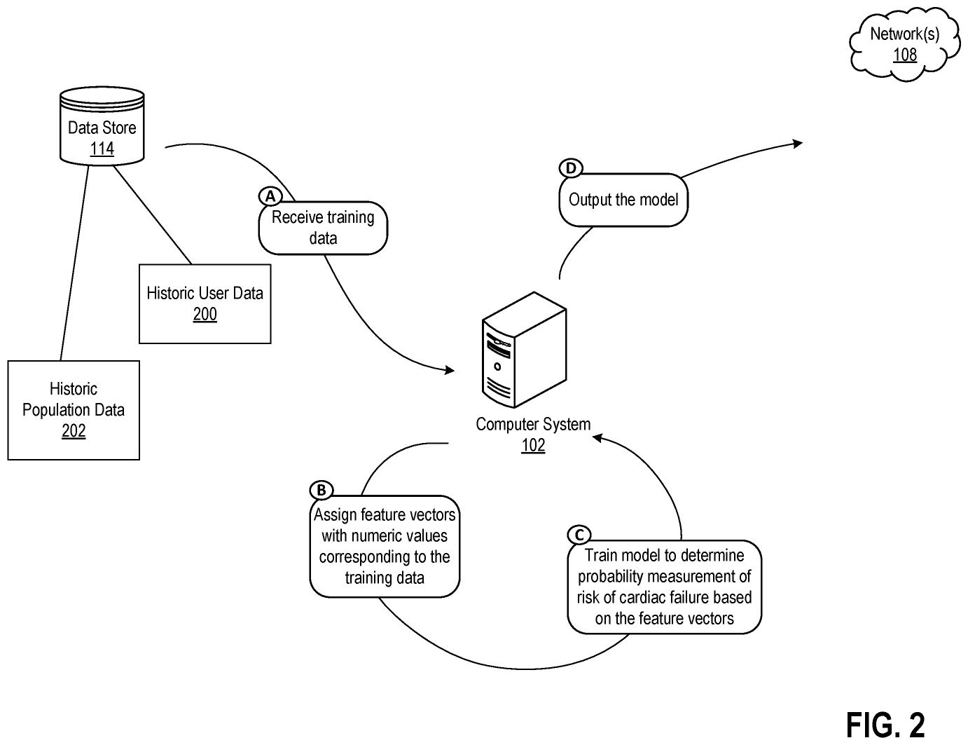

In some implementations, information collected by the pressure sensors can be analyzed by the bed system 100 and/or the computer system 102 to determine various states of the user 106 laying on the bed system 100. As an illustrative example, the computer system 102 can use information collected by the pressure sensors to determine a heartrate or a respiration rate for the user 106. The user can be laying on a side of the bed system 100 that includes an air chamber and the pressure sensors can monitor fluctuations in pressure of the chamber, which can then be used by the computer system 102 to determine the user 106's heartrate and/or respiration rate. More particularly, the computer system 102 can receive pressure data from the pressure sensors, which the computer system 102 uses to generate a time series of cardiac activity and/or respiratory activity for the user 106. The time series can be considered raw data. Then, the computer system 102 can analyze the time series to identify peaks indicative of heartrate changes. The computer system 102 can count the peaks over time (e.g., over a predetermined amount of time) to determine the user 106's heartrate. The computer system 102 can perform a similar process to determine the user 106's respiration rate. The determined heartrate and the determined respiration rate can be features. Thus, the raw data of the time series can be converted into features values, which can then be used by a machine-learning trained model to determine the probability measurement of CHF risk for the user 106. These feature values, along with other feature values, can also be used to train the model using guided learning techniques, as described further in reference to FIG. 2. In some implementations, these feature values may also be used with unguided learning techniques and/or classification techniques to train the model and then use the model during runtime.

As another example, additional processing can be performed using the collected data to determine a sleep state of the user 106 (e.g., awake, light sleep, deep sleep). For example, the computer system 102 can determine when the user 106 falls asleep and, while asleep, the various sleep states (e.g., sleep stages) of the user 106. Based on the determined heartrate, respiration rate, and/or sleep states of the user 106, the computer system 102 can determine sleep fragmentation information and also information about the user 106's objective sleep quality.

Additional information associated with the user 106 of the bed system 100 that can be determined using information collected by the sensors 105A-N includes motion of the user 106, presence of the user 106 on a surface of the bed system 100, weight of the user 106, heart arrhythmia of the user 106, snoring of the user 106 or another user on the bed system 100, and apnea of the user 106. One or more other health conditions of the user 106 can also be determined based on the information collected by the sensors 105A-N described herein. Taking user presence detection for example, the pressure sensors can be used to detect the user 106's presence on the bed system 100 (e.g., via a gross pressure change determination and/or via one or more of a respiration rate signal, heartrate signal, and/or other biometric signals). Detection of the user 106's presence on the bed system 100 can be beneficial to determine, by the computer system 102 information about the user 106's sleep fragmentation and/or overall sleep quality and health.

The user device 112 can be any appropriate type of mobile device of the user 106, including but not limited to a cellphone, smart phone, laptop, tablet, wearable device, or other computing system. The user device 112 may, in some implementations, collect user data that can be used in the processing described further below. The user device 112 can also present a mobile application from which the user 106 can view insights about their sleep and health (e.g., over time, over predetermined periods of time, over a previous night or sleep session, over a current night or sleep session, etc.), as generated/determined by the computer system 102 and based at least in part on data collected by the sensors 105A-N of the bed system 100.

The computer system 102 can be any appropriate type of computing system, computer, network of computers, network of devices, computing device(s), cloud-based server, and/or cloud-based system. The computer system 102 can, in some implementations, be remote from the bed system 100 and configured to generate sleep and/or health insights about users of many bed systems. In some implementations, the computer system 102 can be a controller that is part of the bed system 100 and configured to generate sleep and/or health insights about the user 106 of the bed system 100.

The data store 114 can be a database, cloud-based storage, or other type of repository for storing information/data. The data store 114 can be part of the computer system 102. The data store 114 can also be separate from the computer system 102 and communicably coupled. The data store 114 can store historic data about the user 106 and other users of other bed systems, which can be used for model training and/or runtime determinations of CHF risk in users of the bed systems. The data store 114 can also store training data and machine-learning trained models that are used by the computer system 102 to generate probability measurements of CHF risk in users of the bed systems.

Still referring to FIG. 1, the bed system 100 can collect user data in block A. More particularly, the sensors 105A-N can collect data, such as temperature, pressure, heart rate, respiratory rate, movement, snore, sleep apnea, bed presence, etc. The sensors 105A-N may also collect ballistocardiogram (BCG) signals for the user 106 (e.g., load cells can collect fine-grained weight data indicative of cardiac data for the user 106, pressure sensors can collect pressure changes in air chambers of the bed system 100 that can be indicative of the user 106's biometrics data). The user data can be continuously collected throughout a sleep session of the user 106 (e.g., during the night). This data can also be collected during one or more predetermined periods of time (e.g., when it is detected that the user 106 enters the bed system 100, when it is expected that the user 106 will enter the bed system 100, during certain parts of the user 106's sleep session, etc.). In some implementations, the user device 112 can also collect user data in block A. For example, the user 106 can input data into the mobile application presented at the user device 112, such as their perceived alertness level(s), their perceived sleep quality, a time at which they go to bed, a time at which they wake up or will wake up, and/or biometric data that may be collected by a wearable device and communicated to the user device 112.

The user data collected by the bed system 100 and/or the user device 112 can be transmitted to the computer system 102 in block B. The data can be transmitted in real time, as it is detected. The data can also be transmitted in batches and/or at predetermined times throughout or after the user 106's sleep session. In some implementations, the data can be transmitted at predetermined times, such as once a day, twice a day, once the user 106 wakes up, once the user 106 is detected as exiting the bed system 100, or other times when the user 106 is not detected as being in the bed system 100. In some implementations, the data can also be transmitted to the data store 114 for storage in block B). The data can then be retrieved at a later time by the computer system 102 for use in generating the probability measurement of CHF risk for the user 106 over time.

The computer system 102 can determine sleep fragmentation information for the user based on the user data (block C). Sleep fragmentation information can be determined based on a variety of data, such as data 110. The data 110 can be the user data collected by the bed system 100 and/or the user device 112 in block A. Some of the data 110 may also be historic user data (e.g., specific to the user 106 and/or generic to a population of users) that is stored in the data store 114. Moreover, some of the data 110 may be determined/generated by the computer system 102 and based on other data that is collected by the bed system 100 and/or the user device 112. As an illustrative example, heartrate, sleep disruptions, and/or daytime alertness levels can be determined, by the computer system 102, based on pressure signals detected by the sensors 105A-N. One or more other data can also be determined by the computer system 102 based on data collected by the sensors 105A-N of the bed system 100 and/or the user device 112.

The data 110 used for determining the sleep fragmentation information can include, but is not limited to, sleep detection, wake detection, wake after sleep onset, wake before sleep onset, sleep efficiency, sleep duration, sleep-bout duration, time to fall asleep, arousal index/sleep disruptions, heartrate, heartrate variability, respiratory rate, daytime alertness level(s), demographic factors, blood pressure, and/or inter-beat intervals. Sleep and awake periods can be detected and used to determine fragmentation of the user 106's sleep session. Sleep duration metrics along with wake duration metrics can be informative of sleep fragmentation as they can indicate how long the user 106 is actually asleep and stays asleep during a sleep cycle. Higher sleep duration metrics can allow for sleep stages to occur and therefore support physical and mental development of the user 106. Sleep efficiency metrics can also be informative of sleep fragmentation as they can indicate how much time the user 106 is actually asleep versus how long user 106 is in bed. Higher sleep efficiency can allow for the user 106 to experience restful sleep that may boost overall health wellbeing. Sleep timing metrics can also be informative of sleep fragmentation because consistent sleep timing can improve the user 106's mood and/or alertness throughout a day.

Moreover, wake before sleep onset can be a same or similar measurement as time to fall asleep. The more time it takes the user 106 to fall asleep, the more likely the user 106 may experience sleep fragmentation. The arousal index can indicate a number of disruptions throughout the user 106's sleep cycle and/or sleep session. The more disruptions, the more fragmented the user 106's sleep may be. Heart rate and/or heartrate variability can indicate whether the user 106 experiences disruptive sleep. For example, a heartrate that is significantly higher than a user average can indicate that the user 106 is experiencing poor sleep (or increased sleep fragmentation, for example). Higher respiratory rate can also indicate disruptions in the user 106's sleep. Low daytime alertness levels can indicate disruptions or sleep fragmentation for the user 106. Moreover, demographic factors such as weight, age, gender, and/or body mass index (BMD can also indicate whether the user 106 is experiencing disruptions/sleep fragmentation (e.g., relative to other users having similar demographic factors). Mean Sleep-bout duration data can indicate a dynamic transition of the user between sleep and wake throughout a sleep session/night. Similarly, mean sleep-bout duration data can also be used with the disclosed techniques. Sleep-bout duration data can include times that the user 106 experiences sleep without interruptions. A sleep bout can be considered a portion of uninterrupted sleep within a sleep session. As an illustrative example, a user can sleep through sleep stages N1, N1, and N2 (where N2 is a deeper sleep state than N1 but not as deep as N3), then wake up, and then sleep through sleep stages N1, N1, and N2, before experiencing a second and possibly third wake stage. Each bout of sleeping through sleep stages can vary in lengths of time, including but not limited to 30 seconds, 1 minute, 1.5 minutes, 2 minutes, etc. On average, for example, users can experience approximately 25 minutes of sleep without interruptions. A user, for example, who experiences approximately 5 minutes of sleep before being interrupted may have sleep fragmentation. Similarly, if the user 106's blood pressure is detected as not within expected threshold ranges/amounts, the user 106 may be experiencing sleep fragmentation and, consequently, challenges to their cardiovascular system.

In block D, the computer system 102 can provide the sleep fragmentation information as input to a model. The model can be a model trained using machine learning techniques, as described in reference to FIG. 2. In some implementations, the sleep fragmentation information can be provided to a machine-learning trained algorithm. The model (and/or algorithm) can be trained to take into consideration all the sleep fragmentation information described above in order to determine a probability measurement of CHF risk for users of bed systems.

Moreover, the model can be trained as a population-based model for determining the probability measurement of CHF risk in users over time. The population-based model can be trained using data collected about a variety of different users. The population-based model can also be applied to all users of bed systems and/or a subset or group of users in the population (e.g., users having similar demographic factors, such as weight, BMI, age, and/or gender). The population-based model can be used to determine the probability measurement of CHF risk in the user 106 over long/prolonged periods of time. In some implementations, the population-based model may also be used to determine the probability measurement of CHF risk in the user 106 during a particular sleep session (e.g., during a given night).

The model can also be trained as a personalized model for the particular user 106 and thus trained using user data collected about the particular user 106. The personalized model can be used to determine the probability measurement of CHF risk for the user 106 during a particular sleep session (e.g., on a nightly basis) and/or over time, for prolonged periods of time.

The computer system 102 can receive output from the model indicating the probability measurement of risk of cardiac failure (e.g., CHF) for the user 106 of the bed system 100 (block E). As described herein, the output can include a numeric value indicating the probability measurement of CHF risk. The numeric value can be a dynamic value along a continuum of values, such as 0 to 100, where 0 can be a lowest probability measurement of CHF risk and 100 can be a highest probability measurement of CHF risk. In some implementations, the probability measurement can be a string value, such as low risk, medium risk, and high risk.

The output can be provided at the end of a sleep session of the user 106. For example, the user 106 can go to sleep, and each night, the blocks A-G of FIG. 1 can be performed to determine the user 106's real time CHF risk. In some implementations, the output can also be provided after a predetermined number of sleep sessions and/or at predetermined intervals over a prolonged period of time (e.g., once a week, twice a month, once a month, etc.).

Optionally, the computer system 102 can output the probability measurement and/or perform operation(s) based on the probability measurement in block F. Optionally, the user device 112 can also perform block F. The computer system 102 can output the probability measurement and/or a notification/message/alert about the probability measurement if the probability measurement meets or exceeds some threshold range or value. In some implementations, the computer system 102 may not output the probability measurement and/or the notification/message/alert unless the probability measurement meets or exceeds the threshold range or value. Therefore, the computer system 102 may output the probability measurement if the user is at a high risk of CHF in real-time (e.g., acute CHF) or over some period of time (e.g., chronic CHF).

Optionally, the computer system 102 can output a monthly report indicating the user 106's average CHF risk to the user device 112 and/or a computing device of a relevant healthcare provider. The computer system 102 can also generate one or more other reports about the probability measurement(s) of CHF risk for the user 106 that can be transmitted and presented at the user device 112 and/or the computing device of the relevant healthcare provider. Moreover, the computer system 102 can generate one or more behavioral interventions to help reduce sleep fragmentation for the particular user. The computer system 102 can generate interventions that promote regularity and/or circadian alignment. Such interventions can include recommendations of windows of time by which the user should go to sleep. As an illustrative example, a recommendation can include, but is not limited to, recommending the user go to bed within a 30 minute window and preferably between 10 PM and 11 PM each night. The computer system 102 can also generate interventions such as cognitive behavioral therapy programs in which stimulus control and bed restrictions can be recommended to the user for one or more periods of time (e.g., 2 week period, 3 week period, 6 week period, 8 week period, etc.). One or more other interventions may also be possible.

Optionally, in block F, the computer system 102 can transmit the probability measurement and/or the notification/message/alert to the computing device of the relevant healthcare provider, such as a cardiac physician, the user 106's general physician, a hospital, a nurse, etc. In some implementations, for example, the computer system 102 can determine whether to share the probability measurement with the relevant healthcare provider. The computer system 102 can assess whether the probability measurement exceeds some threshold value/range. Based on how much the probability measurement exceeds the threshold value/range, the computer system 102 may also determine whether to contact emergency healthcare services, such as an emergency room, hospital, ambulance, or first responders.

The computer system 102 can store the probability measurement for the user 106 in the data store 114 (block G). The probability measurement can then be used by the computer system 102 for quantifying CHF risk of the user 106 over prolonged periods of time. The probability measurement can also be used, by the computer system 102, for training the model and/or improving the model over time to more accurately determine probability measurements of CHF risk in the user 106 and/or other users.

FIG. 2 is a conceptual diagram for training a model to determine a probability measurement of congestive heart failure in a user of a bed system. Training can be performed by the computer system 102 described in reference to FIG. 1. In some implementations, training can be performed by another computing system. Training the model can be performed at various times, for example, once a year, twice a year, once a month, etc. In some implementations, the model can be trained and the continuously improved (or improved at one or more predetermined times).

The training techniques described herein can be used to train a model to determine the probability measurement of CHF risk in a particular user. These techniques can also be used to train a model to determine the probability measurement of CHF risk in multiple users or a general population of users. In some implementations, these techniques can be used to train a model to determine the probability measurement of CHF risk in the particular user or a general population of users over extended periods of time. These techniques can also be used to train a model to determine the probability measurement of CHF risk in the particular user or the general population of users for a given night (e.g., sleep session).

Although training in FIG. 2 is described in reference to training a model, the training techniques described herein can also be performed to train more than one model. For example, the techniques can be used to train a model for various groupings of users. The groupings can be defined, for example, based on demographic factors (e.g., age, gender, BMI, weight, geographic location, etc.) or non-demographic factors. The groupings may also vary based on type of data collected and/or analyzed for users. The groupings can also vary based on known health conditions and/or sleep conditions of the users (e.g., sleep apnea, sleep walking, asthma, snore, etc.).

Moreover, the model can be trained using machine learning techniques including but not limited to logistic regression, linear regression, deep neural networks, and decision trees. Logistic regression is a process of modeling probability of a discrete outcome given one or more input variables. A logistic regression model can output a binary value, such as 0 or 1, true or false, and/or yes or no. In such a scenario, the computer system 102 can normalize the output from the regression model by multiplying the output by a predetermined factor to generate the probability measurement of CHF risk. Deep neural networks can be used to identify underlying relationships in a set of data. Such networks can adapt to changing input to generate best, preferred or optimal output. Example deep neural networks that can be used with the disclosed training techniques can include, but are not limited to, a multiplayer perception (MLP) model, convolutional neural network (CNN) model, and/or recurrent neural network (RNN) model. Decision trees are forms of supervised machine learning in which data is continuously split according to one or more defined parameters. A tree can include decision nodes and leaves, the leaves being decisions or final outcomes and the decision nodes being where the data is split. One or more types of decision trees can be used, including but not limited to classification trees and regression trees. Although various machine learning techniques can be used, the training techniques of FIG. 2 are described in terms of logistic regression modeling, which includes the use of feature vectors.

Referring to FIG. 2, the computer system 102 can receive training data from the data store 114 in block A. In some implementations, the computer system 102 can receive user data collected by bed systems directly from the bed systems, as described in blocks A-B of FIG. 1. The training data can include historic user data 200 and/or historic population data 202. The historic user data 200 and the historic population data 202 can be collected over time by sensors of bed systems and/or user devices. Moreover, the data 200 and 202 can include probability measurements of CHF risk as previously determined by the computer system 102. The historic user data 200 and/or the historic population data 202 can include any of the data described throughout this disclosure, including but not limited to sleep detection, wake detection, wake after sleep onset, wake before sleep onset, sleep efficiency, sleep duration, sleep-bout duration, time to fall asleep, arousal index/sleep disruptions, HR, HRV, RR, daytime alertness level(s), demographic factors, blood pressure, IBIs, and/or other BCG signals.

In some implementations, some of the training data received in block A can be annotated and labeled (e.g., by a user and/or by a computing system). For example, low daytime alertness levels can be labeled as indicative of some level of sleep fragmentation. As another example, certain blood pressure data can be labeled as indicative of some level of sleep fragmentation based on the blood pressure data exceeding threshold ranges and/or values. Additionally or alternatively, combinations of training data can be labeled and annotated as indicative of various levels of sleep fragmentation.

For training purposes, the computer system 102 can assign feature vectors with numeric values that correspond to the training data in block B. Feature vectors can be used to represent numeric or symbolic characteristics, or features, of an object. In the context of CHF risk, each feature vector can represent a different metric used to quantify sleep fragmentation and, consequently, the CHF risk. As an illustrative example, a feature vector can be assigned values of a user's heartrate over time, another feature vector can be assigned values of how long it takes the user to fall asleep, and another feature vector can be assigned values of how many disruptions the user experiences during sleep sessions. As another example, feature vectors can be assigned values including but not limited to a total amount of minutes for wake after sleep onset, a percentage of sleep efficiency, a total amount of minutes of sleep duration, a total amount of minutes of sleep-bout duration, and cardiovascular values such as heartrate as beats per minute and respiratory rate as breaths per minute. In some implementations, the values for the feature vectors can be mapped into n-dimensional space, where each dimension may represent a different feature. Clusters of values can be identified as indicators of sleep fragmentation. The model can then be trained to correlate such indicators of sleep fragmentation with CHF risk.

In block C, the computer system 102 can train a model to determine the probability measurement of risk of cardiac failure (e.g., CHF risk) based on the feature vectors. As mentioned above, the computer system 102 can train the model to correlate clusters of sleep fragmentation indicators (or feature vector values) with CHF risk.

Output from a logistic or linear regression model can include a binary value, such as 0 or 1, where 0 can indicate no CHF risk and 1 can indicate CHF risk. In some implementations, instead of a binary value, the model output can be a real value between 0 and 1. The computer system 102 can multiply the output by a numeric factor to generate an integer value that quantifies CHF risk. For example, the computer system 102 can multiply the output by a factor of 100 to quantify the CHF risk as a probability measurement. The computer system 102 can also multiply the output by one or more other factors to quantify the CHF risk as a probability measurement.

Output from a deep neural network can include an integer value, such as a value on a continuum or scale, like 0 to 100. Output from a decision tree can include a binary value (e.g., numeric, string, Boolean) and can also be multiplied by a factor to quantify the CHF risk as a probability measurement. In some implementations, output from the decision tree can also include a probability measurement of CHF risk.

The computer system 102 can then output the model in block D. Outputting the model can include storing the model in the data store 114 for future retrieval and use during runtime. Outputting the model can also include storing the model in local memory such that the model can be used during runtime for real-time CHF risk determinations.

In some implementations, the model can be transmitted to a controller of a bed system, such as the bed system 100 in FIG. 1. The controller of the bed system can then be configured to collect user data collected by sensors of the bed system and provide the collected user data to the model to determine the probability measurement of CHF risk for the user of the bed system. Therefore, the controller of the bed system, rather than a remote computer such as the computer system 102, can determine the probability measurement of CHF risk for the user. In yet some implementations, the model can be transmitted to the user's device. The user's device, instead of the computer system 102, can then receive the user data and provide the user data as input to the model to determine the probability measurement of CHF risk for the user.

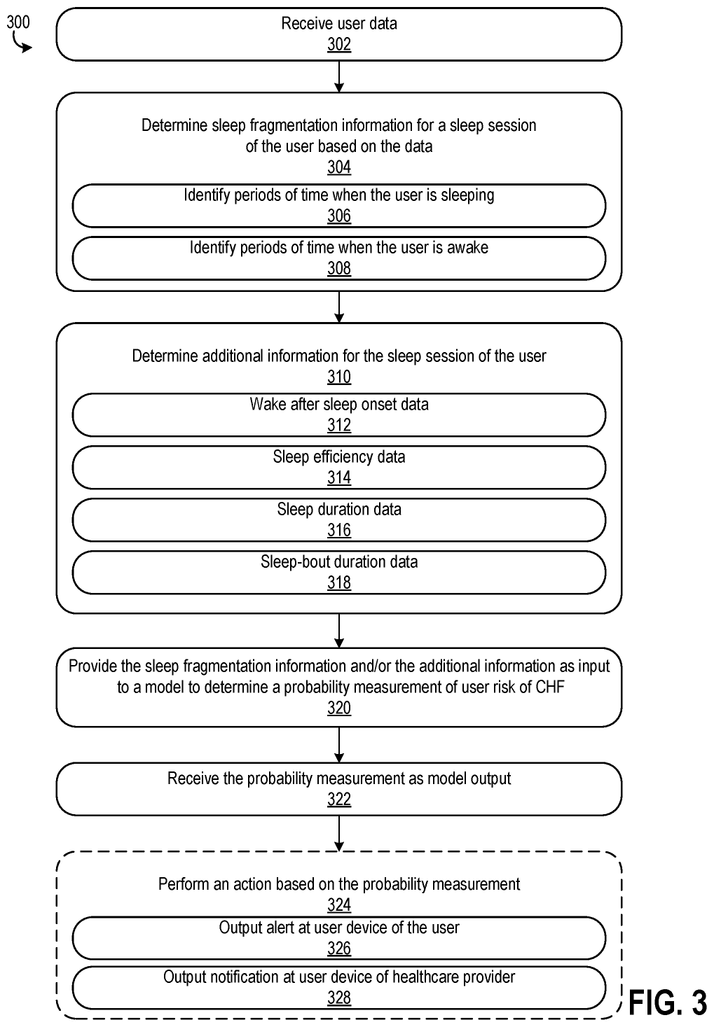

FIG. 3 is a flowchart of a process 300 for determining a probability measurement of congestive heart failure in a user of a bed system. The process 300 can be performed during or after a sleep session of the user. The process 300 can be performed during and/or after each sleep session of the user. The process 300 can also be performed at predetermined times and/or over prolonged periods of time (e.g., every other sleep session, a couple times a week, once a day, etc.). Accordingly, the process 300 can be performed to determine the probability measurement of CHF risk in the patient for a given/particular night as well over prolonged periods of time.

The process 300 can be performed by the computer system 102. The process 300 can also be performed by one or more other computing systems, devices, computers, networks, cloud-based systems, cloud-based services, bed controllers, and/or user devices. For illustrative purposes, the process 300 is described from the perspective of a computer system.

Referring to the process 300, the computer system can receive user data in block 302. As described herein, the computer system can receive the user data collected by sensors of a bed system when the user rests on the bed system. Refer to blocks A-B in FIG. 1 for additional discussion about the user data and receiving the user data.

In block 304, the computer system can determine sleep fragmentation information for a sleep session of the user based on analyzing the data. This can include identifying periods of time when the user is sleeping during the sleep session in block 306. Additionally or alternatively, this can include identifying periods of time when the user is awake during the sleep session in block 308. Therefore, the computer system can determine the sleep fragmentation information as a function of the sensed user data from at least one sensor of the user on the bed system.

The computer system can process the user data, such as pressure data, to determine when the user enters the bed system and when they fall asleep. Periods of time when the user is sleeping can be detected from pressure data that indicates a change in heartrate or other BCG signals. For example, if the user's heartrate slows to or below a threshold amount, the computer system can determine that the user has fallen asleep. Similarly, the computer system can process pressure data to determine when the user may be snoring, which further can be used to identify periods of time when the user is sleeping. One or more other data received in block 302 can be processed to determine the sleep fragmentation information for the sleep session of the user. Refer to block C in FIG. 1 for additional discussion about determining the sleep fragmentation information.

In block 310, the computer system can determine additional information for the sleep session of the user. The additional information can include, but is not limited to, wake after sleep onset data (block 312), sleep efficiency data (block 314), sleep duration data (block 316), and sleep-bout duration data (block 318). For example, the computer system can determine wake after sleep onset data as a function of the sensed user data of the user from the at least one sensor of the bed (block 312). The computer system can also determine sleep efficiency data as a function of the sensed user data (block 314). The computer system can also determine sleep duration data as a function of the sensed user data (block 316). The computer system can determine sleep-bout duration data as a function of the sensed user data (block 318). In some implementations, the computer system can determine less than all the data in blocks 312-318. As an illustrative example, the computer system may only determine the wake after sleep onset data, the sleep efficiency data, and the sleep-bout duration data. Any other combination of the data in blocks 312-318 can be determined and used by the computer system to determine the probability measurement of CHF risk for the user.

One or more of the blocks 312-318 can be determined based on processing the data received in block 302. Additionally or alternatively, one or more of the blocks 312-318 can be determined based on the sleep fragmentation information determined in blocks 304-308. In some implementations, the data in blocks 312-318 can be used to determine the sleep fragmentation information in blocks 304-308. Therefore, block 310 can be performed before block 304 and/or as part of determining the sleep fragmentation information in blocks 304-308.

Next, in block 320, the computer system can provide the sleep fragmentation information and/or the additional information as input to a model to determine a probability measurement of user risk of CHF. The model could have been trained to determine a probability measurement of a risk of cardiac failure (e.g., risk of CHF) for users of bed systems based at least in part on user data associated with the users. In some implementations, the model can be trained to correlate at least one of the additional data from blocks 310-318 with the sleep fragmentation information from blocks 304-308 to quantify the probability measurement of CHF risk. The model can also calculate the probability measurement as a function of the determined sleep fragmentation information. Sometimes, the model can calculate the probability measurement of CHF risk as a function of the determined wake after sleep onset data, sleep efficiency data, and sleep-bout duration data. The model can also be trained to calculate the probability measurement as a function of a combination of any of the determined additional data in blocks 312-318. The model can be trained using the techniques described in FIG. 2.

In some implementations, block 320 can include selecting, by the computer system, an appropriate model from a data store (e.g., the data store 114) of preexisting models for determining the probability measurement of CHF risk for the user. The computer system can select the model based on one or more factors, including but not limited to user information, demographic factors, whether the computer system is determining the probability measurement for a current or given sleep session/night, whether the computer system is determining the probability measurement for an extended period of time, etc. Thus, multiple models can be generated and trained to determine the probability measurement of CHF risk then stored in the data store for runtime selection, retrieval, and use. In some implementations, as described in reference to FIG. 1, a population-based model can be trained and used to determine the probability measurement of CHF risk in various different users. Refer to block D in FIG. 1 for additional discussion about determining the probability measurement of CHF risk using the model.

In block 322, the computer system can receive the probability measurement as model output. As described in reference to FIGS. 1-2, the model output can be a numeric value indicating a probability that the user has a risk of CHF. The model output can also be a binary value, such as 0 to 1, especially when the model is a logistic or linear regression model. The computer system can then normalize or adjust this binary value by multiplying the binary value by a factor, such as 100, to quantify the CHF risk as a probability measurement. In some implementations, the model output can be a Boolean value, such as true/false. The model output can also be a string value, such as CHF risk/no CHF risk. For example, the model output can include low risk, medium risk, and high risk. One or more other values can be generated and outputted by the model. Refer to block E in FIG. 1 and block C in FIG. 2 for additional discussion about model output.

In block 324, the computer system can optionally perform an action based on the probability measurement. In other words, the computer system can output a signal to perform an action based on the calculated probability measurement of CHF risk for the user. The computer system can output an alert at a user device of the user in block 326. If, for example, the probability measurement exceeds some threshold value (e.g., the probability measurement is greater than 90 on a scale of 0-100), the computer system can generate the alert notifying the user that they are at high risk of CHF. In some implementations, the computer system may only output the alert or some other notification/message at the user device of the user if the probability measurement exceeds some threshold value. The alert or other notification/message can be presented in a mobile application at the user device, as described in reference to FIG. 1.

The computer system can also output a notification at a user device of a relevant healthcare provider in block 328. For example, if the probability measurement exceeds some threshold value, the computer system can generate the notification, which can also be an alert, notifying the relevant healthcare provider of the user's CHF risk. In some implementations, the computer system may optionally generate an alert or other form of contact to an emergency medical service, such as an ambulance, first responder, and/or emergency room if the probability measurement exceeds another threshold value (e.g., the probability measurement is greater than 95 on the scale of 0-100).

In block 324, the computer system can optionally perform one or more other actions. For example, the computer system can store the probability measurement in the data store. The probability measurement can then be used for future training and/or continuous improvements of the model or models. The computer system can also generate a report about the probability measurement and/or the user's probability measurements over one or more periods of time. The reports can be generated on a daily, weekly, monthly, yearly, etc. basis. Similarly, the probability measurement can be used by the computer system to analyze the user's health and cardiovascular activity over time to generate various other reports and CHF risk assessments for the user. Refer to blocks F-G in FIG. 1 for additional discussion.

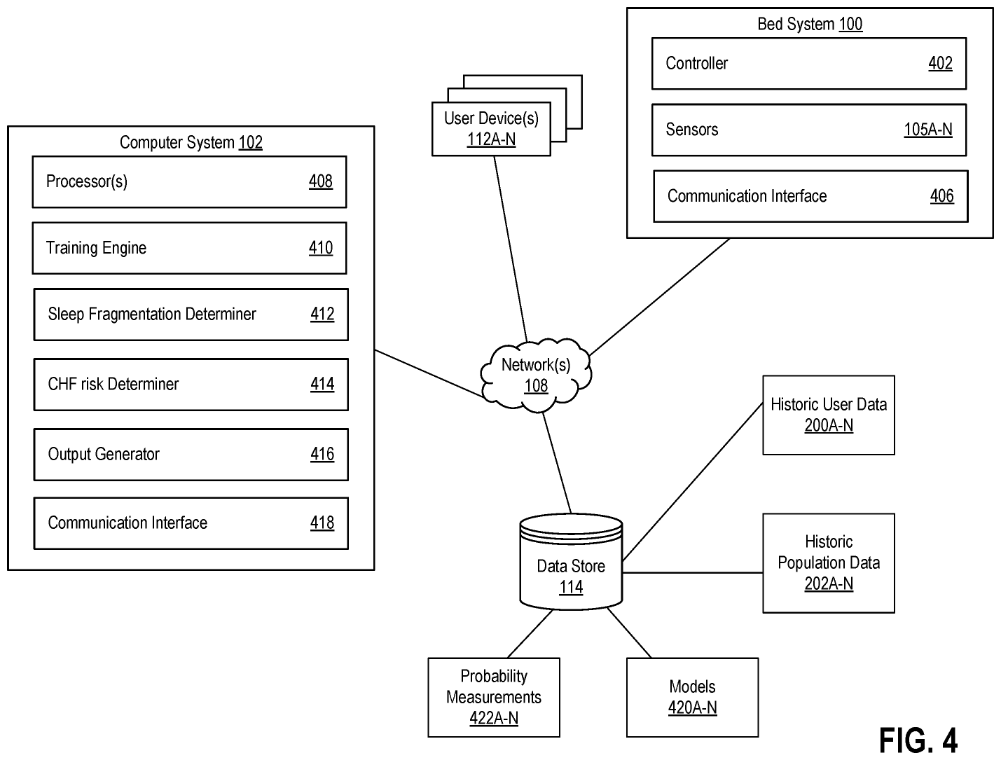

FIG. 4 is a system diagram of one or more hardware and software components that can be used to perform the disclosed techniques. As described throughout this disclosure, the computer system 102, user devices 112A-N, bed system 100, and data store 114 can communicate via the network(s) 108.

The bed system 100 can include a controller 402, sensors 105A-N, and communication interface 406. Refer to FIG. 1 for additional discussion about the bed system 100 and the sensors 105A-N. Moreover, the controller 402 can be configured to control one or more components of the bed system 100. In some implementations, the controller 402 can include processors that can execute instructions causing the controller 402 to perform one or more of the techniques described herein. For example, the controller 402 can perform one or more functions of the computer system 102, such as determining sleep fragmentation information for a user of the bed system 100 and determining a probability measurement of CHF risk for the user. Additionally, the communication interface 406 can provide for communication between the components of the bed system 100 and one or more other components described herein.

The computer system 102 can include processor(s) 408, training engine 410, sleep fragmentation determiner 412, CHF risk determiner 414, output generator 416, and communication interface 418. The processor(s) 408 can be configured to perform one or more of the operations described throughout this disclosure. The training engine 410 can be configured to train models using machine learning techniques to determine the probability measurement of CHF risk in users of the bed systems. Refer to FIG. 2 for additional discussion about training operations that can be performed by the training engine 410. In brief, for example, the training engine 410 can retrieve the historic user data 200A-N and/or the historic population data 202A-N from the data store 114. The training engine 410 can use the retrieved data to train a model, which can be stored as models 420A-N in the data store 114.

During runtime, the sleep fragmentation determiner 412 can receive user data collected by the sensors 105A-N of the bed system 100 during the user's sleep session. The determiner 412 can process this information, as described in reference to FIGS. 1 and 3, to determine the sleep fragmentation information.

The CHF risk determiner 414 can be configured to retrieve the model 420A-N from the data store 114. User data collected by the sensors 105A-N and/or sleep fragmentation information determined by the sleep fragmentation determiner 412 can be provided as input to the retrieved model 420A-N so at the CHF risk determiner 414 to generate output indicating the probability measurement of CHF risk for the user of the bed system 100. The probability measurement of the CHF risk for the user can then be stored in the data store 114 as probability measurements 422A-N.

The output generator 416 can be configured to generate output based on the probability measurement of the CHF risk for the user. Refer to FIGS. 1 and 3 for additional discussion about the output that can be generated. The output can be transmitted to the user device 112A-N of the user of the bed system 100. The output can also be transmitted to user devices 112A-N of healthcare providers, as described herein. Finally, the communication interface 418 can provide for communication between the computer system 102 and one or more other components described herein.

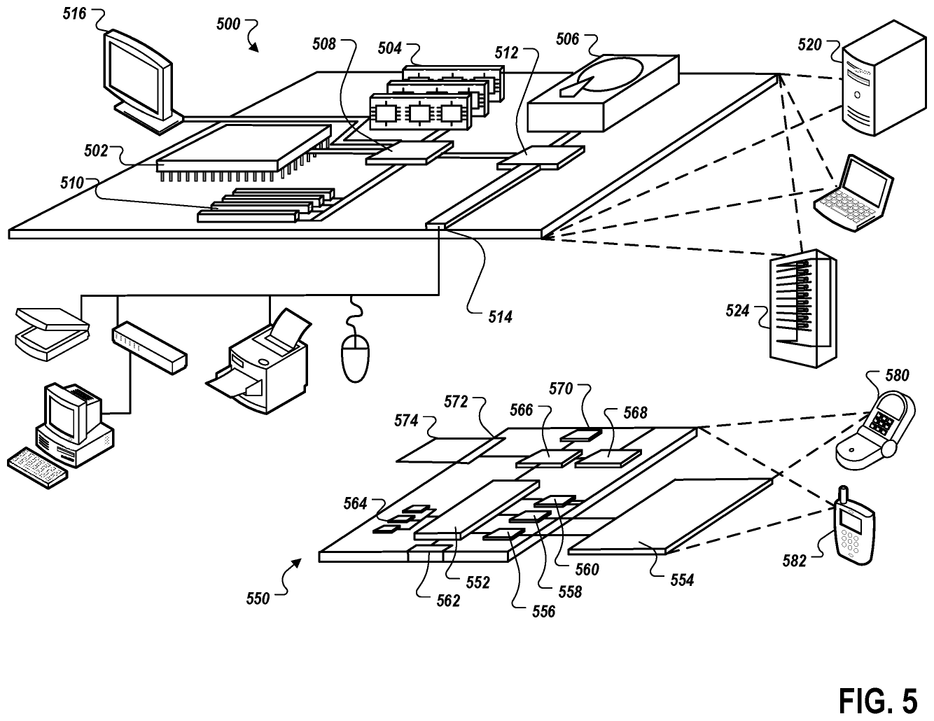

FIG. 5 shows an example of a computing device 500 and an example of a mobile computing device that can be used to implement the techniques described here. The computing device 500 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and can be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 504 stores information within the computing device 500. In some implementations, the memory 504 is a volatile memory unit or units. In some implementations, the memory 504 is a non-volatile memory unit or units. The memory 504 can also be another form of computer-readable medium, such as a magnetic or optical disk.

The storage device 506 is capable of providing mass storage for the computing device 500. In some implementations, the storage device 506 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 504, the storage device 506, or memory on the processor 502.

The high-speed interface 508 manages bandwidth-intensive operations for the computing device 500, while the low-speed interface 512 manages lower bandwidth-intensive operations. Such allocation of functions is exemplary only. In some implementations, the high-speed interface 508 is coupled to the memory 504, the display 516 (e.g., through a graphics processor or accelerator), and to the high-speed expansion ports 510, which can accept various expansion cards (not shown). In the implementation, the low-speed interface 512 is coupled to the storage device 506 and the low-speed expansion port 514. The low-speed expansion port 514, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 500 can be implemented in a number of different forms, as shown in the figure. For example, it can be implemented as a standard server 520, or multiple times in a group of such servers. In addition, it can be implemented in a personal computer such as a laptop computer 522. It can also be implemented as part of a rack server system 524. Alternatively, components from the computing device 500 can be combined with other components in a mobile device (not shown), such as a mobile computing device 550. Each of such devices can contain one or more of the computing device 500 and the mobile computing device 550, and an entire system can be made up of multiple computing devices communicating with each other.

The mobile computing device 550 includes a processor 552, a memory 564, an input/output device such as a display 554, a communication interface 566, and a transceiver 568, among other components. The mobile computing device 550 can also be provided with a storage device, such as a micro-drive or other device, to provide additional storage. Each of the processor 552, the memory 564, the display 554, the communication interface 566, and the transceiver 568, are interconnected using various buses, and several of the components can be mounted on a common motherboard or in other manners as appropriate.

The processor 552 can execute instructions within the mobile computing device 550, including instructions stored in the memory 564. The processor 552 can be implemented as a chip set of chips that include separate and multiple analog and digital processors. The processor 552 can provide, for example, for coordination of the other components of the mobile computing device 550, such as control of user interfaces, applications run by the mobile computing device 550, and wireless communication by the mobile computing device 550.

The processor 552 can communicate with a user through a control interface 558 and a display interface 556 coupled to the display 554. The display 554 can be, for example, a TFT (Thin-Film-Transistor Liquid Crystal Display) display or an OLED (Organic Light Emitting Diode) display, or other appropriate display technology. The display interface 556 can comprise appropriate circuitry for driving the display 554 to present graphical and other information to a user. The control interface 558 can receive commands from a user and convert them for submission to the processor 552. In addition, an external interface 562 can provide communication with the processor 552, so as to enable near area communication of the mobile computing device 550 with other devices. The external interface 562 can provide, for example, for wired communication in some implementations, or for wireless communication in other implementations, and multiple interfaces can also be used.

The memory 564 stores information within the mobile computing device 550. The memory 564 can be implemented as one or more of a computer-readable medium or media, a volatile memory unit or units, or a non-volatile memory unit or units. An expansion memory 574 can also be provided and connected to the mobile computing device 550 through an expansion interface 572, which can include, for example, a SIMM (Single In Line Memory Module) card interface. The expansion memory 574 can provide extra storage space for the mobile computing device 550, or can also store applications or other information for the mobile computing device 550. Specifically, the expansion memory 574 can include instructions to carry out or supplement the processes described above, and can include secure information also. Thus, for example, the expansion memory 574 can be provide as a security module for the mobile computing device 550, and can be programmed with instructions that permit secure use of the mobile computing device 550. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 564, the expansion memory 574, or memory on the processor 552. In some implementations, the computer program product can be received in a propagated signal, for example, over the transceiver 568 or the external interface 562.

The mobile computing device 550 can communicate wirelessly through the communication interface 566, which can include digital signal processing circuitry where necessary. The communication interface 566 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 568 using a radio-frequency. In addition, short-range communication can occur, such as using a Bluetooth, WiFi, or other such transceiver (not shown). In addition, a GPS (Global Positioning System) receiver module 570 can provide additional navigation- and location-related wireless data to the mobile computing device 550, which can be used as appropriate by applications running on the mobile computing device 550.

The mobile computing device 550 can also communicate audibly using an audio codec 560, which can receive spoken information from a user and convert it to usable digital information. The audio codec 560 can likewise generate audible sound for a user, such as through a speaker, e.g., in a handset of the mobile computing device 550. Such sound can include sound from voice telephone calls, can include recorded sound (e.g., voice messages, music files, etc.) and can also include sound generated by applications operating on the mobile computing device 550.

The mobile computing device 550 can be implemented in a number of different forms, as shown in the figure. For example, it can be implemented as a cellular telephone 580. It can also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular embodiments of particular disclosed technologies. Certain features that are described in this specification in the context of separate embodiments can also be implemented in combination in a single embodiment in part or in whole. Conversely, various features that are described in the context of a single embodiment can also be implemented in multiple embodiments separately or in any suitable sub combination. Moreover, although features may be described herein as acting in certain combinations and/or initially claimed as such, one or more features from a claimed combination can in some cases be excised from the combination, and the claimed combination may be directed to a sub combination or variation of a sub combination. Similarly, while operations may be described in a particular order, this should not be understood as requiring that such operations be performed in the particular order or in sequential order, or that all operations be performed, to achieve desirable results. Particular embodiments of the subject matter have been described. Other embodiments are within the scope of the following claims.

What is claimed is:

1. A method for determining a probability measurement of a risk of cardiac failure for a user of a bed system, the method comprising:
receiving, by a computer system, user data collected by sensors of a bed system when a user rests on the bed system;
determining, by the computer system, sleep fragmentation information for a sleep session of the user based on analyzing the user data;
providing, by the computer system, the sleep fragmentation information as input to a model that was trained to determine a probability measurement of a risk of cardiac failure for users of bed systems based at least in part on user data associated with the users;
receiving, by the computer system, the probability measurement of the risk of cardiac failure for the user of the bed system as output from the model; and
performing, by the computer system, an action based on the probability measurement.

2. The method of claim 1, wherein the user data includes ballistocardiogram (BCG) signals.

3. The method of claim 1, wherein the model was trained with a training dataset of historic user data associated with the user.

4. The method of claim 1, wherein the model was trained with a training dataset of historic user data associated with different users of the bed systems.

5. The method of claim 1, wherein the model is a logistic regression model.

6. The method of claim 1, wherein the model was trained, by the computer system, using feature vectors that were assigned numeric values corresponding to training user data.

7. The method of claim 6, wherein the training user data includes at least one of wake time after sleep onset, sleep efficiency, sleep duration, sleep-bout duration, wake time before sleep onset, quantity of sleep disruptions, heartrate, heartrate variability, respiratory rate, or daytime alertness levels.

8. The method of claim 6, wherein the training user data includes demographics information, the demographics information including at least one of weight, age, gender, or body mass index (BMI).

9. The method of claim 1, wherein determining, by the computer system, sleep fragmentation information for the user comprises identifying periods of time during the sleep session when the user is sleeping and periods of time during the sleep session when the user is awake.

10. The method of claim 1, further comprising:
providing, by the computer system as input to the model, at least one of (i) wake after sleep onset data, (ii) sleep efficiency data, (iii) sleep duration data, or (iv) sleep-bout duration data,
wherein the model was trained, by the computer system, to correlate at least one of (i)-(iv) with stages of sleep fragmentation for the users of the bed systems.

11. The method of claim 1, wherein the probability measurement of the risk of cardiac failure is a numeric value.

12. The method of claim 1, wherein:
the output from the model is a value between 0 and 1,
the method further comprising: multiplying, by the computer system, the value with a numeric factor to generate a score of the risk of cardiac failure.

13. The method of claim 1, wherein the probability measurement of the risk of cardiac failure is a categorical value, the categorical value being at least one of low risk, medium risk, or high risk.

14. The method of claim 1, wherein performing, by the computer system, an action based on the probability measurement comprises outputting an alert at a user device of the user indicating that the user is at risk of cardiac failure based on a determination that the probability measurement exceeds a threshold range.

15. The method of claim 1, wherein performing, by the computer system, an action based on the probability measurement comprises transmitting a notification to a user device of a healthcare provider that the user is at risk of cardiac failure based on a determination that the probability measurement exceeds a threshold range.

16. The method of claim 1, wherein the cardiac failure is congestive heart failure.

17. A system for determining a probability measurement of a risk of cardiac failure for a user of a bed system, the system comprising:
a bed having at least one sensor; and
a computer system in communication with the at least one sensor of the bed, the computer system configured to:
receive, from the at least one sensor, user data collected by the at least one sensor when a user rests on the bed;
determine sleep fragmentation information for a sleep session of the user based on analyzing the user data;
provide the sleep fragmentation information as input to a model that was trained to determine a probability measurement of a risk of cardiac failure for users of beds based at least in part on user data associated with the users;
receive the probability measurement of the risk of cardiac failure for the user of the bed as output from the model; and
perform an action based on the probability measurement.

18. The system of claim 17, wherein:
the bed further comprises a controller, and
the computer system is the controller.

19. The system of claim 17, wherein the computer system is remote from the bed.

20. A system for determining a probability measurement of a risk of cardiac failure for a user of a bed system, the system comprising:
a bed having at least one sensor; and
a computer system in communication with the at least one sensor of the bed, the computer system configured to:
receive, from the at least one sensor, user data collected by the at least one sensor when a user rests on the bed;
determine sleep fragmentation information for a sleep session of the user based on analyzing the user data;
determine at least one of (i) wake after sleep onset data, (ii) sleep efficiency data, (iii) sleep duration data, and (iv) sleep-bout duration data based on analyzing the user data;
provide the sleep fragmentation information and at least one of (i)-(iv) as input to a model that was trained to determine a probability measurement of a risk of cardiac failure for users of beds based at least in part on user data associated with the users;
receive the probability measurement of the risk of cardiac failure for the user of the bed as output from the model; and
perform an action based on the probability measurement.
